(19) United States
(12) Patent Application Publication
(10) Pub. No.: US 2012/0252078 A1
(43) Pub. Date: Oct. 4, 2012

(54) Microorganism Which Produces L-Amino Acid and Method for Producing L-Amino Acid Using the Same

(75) Inventors: Jae Yeong Ju, Seongnam-si (KR); Kwang Ho Lee, Daejeon (KR); Hyun Ae Bae, Seoul (KR)

(73) Assignee: CJ Cheiljetdang Corporation, Seoul (KR)

(21) Appl. No.: 13/254,078

(22) PCT Filed: Feb. 16, 2010
(86) PCT No.: PCT/KR2010/000933

§ 371 Date: Nov. 21, 2011

(30) Foreign Application Priority Data
Mar. 3, 2009 (KR) 10-2009-0018128

(51) Int. Cl. C12P 13/04; C12P 13/08; C12N 1/21

(52) U.S. Cl. 435/115; 435/252.33; 435/106

(57) ABSTRACT

The present invention relates to a microorganism belonging to the genus Escherichia sp. and a method for producing L-amino acid using the same. The microorganism belonging to the genus Escherichia sp. with sucrose assimilability and L-amino acid producing ability is obtained by introducing a gene encoding a sucrose assimilative microorganism-derived sucrose metabolic enzyme to the sucrose non-assimilative microorganism belonging to the genus Escherichia sp. having an L-amino acid producing ability.

### BACKGROUND OF THE INVENTION

1. Field of the Invention

The present invention relates to a microorganism belonging to the genus Escherichia sp. having a sucrose assimilability and an L-amino acid producing ability, which is obtained by introducing a gene encoding a sucrose assimilative microorganism-derived sucrose metabolic enzyme to a sucrose non-assimilative microorganism belonging to the genus Escherichia sp. having an L-amino acid producing ability, and a method for producing an L-amino acid using the same.

2. Description of the Related Art

Due to the growing demand for bio-fuel production and crop failures caused by unusual climate, the price of starch sugar used as a main carbon source in industrial fermentation has rapidly increased. Alternatively, the use of sucrose or molasses containing a high concentration of sucrose, cheaper than starch sugar, as a carbon source in industrial fermentation, is advantageous ensure cost competitiveness.

Approximately 50% of wild-type naturally occurring E. coli is able to metabolize sucrose, but E. coli K12 strain, B strain, and C strain usually used in industrial fermentation, have no ability to assimilate sucrose (Mol. Microbiol, (1998) 2:1-8, Can. J. Microbiol, (1999) 45:418-422). Therefore, one of the most important challenges in the fermentation industry is the identification of genes involved in sucrose assimilation, the establishment of enhanced sucrose assimilation-related genes by improvement, and the application of the genes to the sucrose non-assimilative, industrial E. coli strains for the production of desired metabolites.

To impart a sucrose-assimilability to industrial E. coli strains, methods of introducing genes or gene cluster involved in sucrose assimilation, derived from microorganisms having a sucrose-assimilability have been generally used. For example, a method of imparting sucrose-assimilability to E. coli K12 by transformation with the scr regulon that is present in the species Salmonella belonging to the family Enterobacteriaceae (J. Bacteriol. (1982) 151:68-76, Mol. Microbiol. (1998) 2:1-8, J. Bacterial. (1991) 173:7464-7470, U.S. Pat. No. 7,179,623), Klebsiella pneumoniae (J. Gen. Microbiol, (1988) 134:1635-1644), and Erwinia amylovora (J. Bacteriol. (2000) 182:5351-5358) has been well known in the art. Introduction of the csc regulon derived from non-K12 E. coli or pathogenic E. coli having the sucrose-assimilability (Appl. Environ. Microbiol, (1992) 58:2081-2088, U.S. Pat. No. 6,960,455), introduction of gene cluster involved in sucrose assimilation that is present in conjugative plasmid scr53 isolated from E. coli AB1281 (U.S. Pat. No. 4,806,480), and introduction of scr regulon and sac operon derived from Gram-positive microorganisms, Streptococcus mutans (J. Bacteriol, (1989) 171:263-271) and Bacillus subtilis (J. Bacteriol, (1989) 171:1519-1523) are also known. U.S. Pat. No. 7,179,623 discloses a method of producing lysine, isoleucine and valine using E. coli K12 that is prepared by introducing an E. coli VKPM B-7915-derived scr regulon thereto.

However, there is still a need of an industrial microorganism having an efficient sucrose utilization system and a fermentation method using the same. Therefore, the present inventors found that an L-amino acid can be produced from sucrose at a high yield using an L-amino acid-producing microorganism belonging to the genus Escherichia sp. microorganism, which is prepared by introducing a gene cluster involved in sucrose assimilation, derived from a sucrose assimilative Streptococcus mutans, thereby completing the present invention.

### SUMMARY OF THE INVENTION

An object of the present invention is to provide a microorganism belonging to the genus Escherichia sp. having sucrose assimilability and an L-amino acid producing ability, which is prepared by imparting sucrose assimilability to a sucrose non-assimilative microorganism belonging to the genus Escherichia sp. having an L-amino acid producing ability.

Another object of the present invention is to provide a method for producing an L-amino acid from sucrose using the microorganism belonging to the genus Escherichia sp. having sucrose assimilability and an L-amino acid producing ability.

### BRIEF DESCRIPTION OF THE DRAWINGS

FIG. 1 shows the construction of a recombinant plasmid pAscrSM containing Streptococcus mutans-derived scrKABR according to one specific embodiment of the present invention.

### DETAILED DESCRIPTION OF THE PREFERRED EMBODIMENTS

In order to achieve the above objects, the present invention provides a microorganism belonging to the genus Escherichia sp. having a sucrose assimilability and an L-amino acid producing ability, which is obtained by introducing a gene encoding a sucrose assimilative microorganism-derived sucrose metabolic enzyme to a sucrose non-assimilative a microorganism belonging to the genus Escherichia sp. having an L-amino acid producing ability.

As used herein, the term “sucrose non-assimilative microorganism” means a microorganism that cannot utilize sucrose as a carbon source, and the term “microorganism having sucrose assimilability and an L-amino acid producing ability” means a microorganism that can metabolize sucrose as a carbon source so as to produce an L-amino acid.

As used herein, the term “sucrose metabolic enzyme” means an enzyme required for utilization of sucrose as a carbon source, and it includes fructokinase, sucrose PTS permease, sucrose hydrolase, invertase or the like, but is not limited thereto.

In the present invention, the “sucrose metabolic enzyme” is also called “Scr-PTS enzyme”.

A metabolic system utilizing sucrose as a carbon source can be largely divided into PTS (phosphoenolpyruvate dependent sucrose phosphotransferase)-based sucrose metabolic system (Scr-PTS system) and non-PTS-based sucrose metabolic system (Scr-non PTS system) according to a phosphate source for phosphorylation of influent sucrose in a cell. Most microorganisms capable of utilizing sucrose have the Scr-PTS system.

A representative example of the PTS-based Scr-PTS system using phosphoenolpyruvate (PEP) as a phosphate source for phosphorylation of sucrose includes a conjugative plasmid pUR400 of Gram-negative Salmonella typhimurium, a scr regulon present on the chromosome of Klebsiella pneumoniae or the like. The scr regulon is composed of 5 genes, scrK (fructokinase), scrY (sucrose porin), scrA (sucrose-specific EIIBC component), scrB (sucrose-6-phosphate hydrolase) and scrR (LacI-related sucrose-specific repressor), and two operons, scrK and scrYAB are negatively controlled by the ScrR repressor (Mol. Microbiol. (1993) 9:195-209). According to a mechanism of the scr regulon, external sucrose is transported into a periplasmic space through an outer membrane protein (OMP), ScrY. The transported sucrose is transported into a cell in the form of sucrose-6-phosphate through a sucrose PTS cycle including ScrA. Sucrose-6-phosphate is then hydrolyzed to glucose-6-phosphate and fructose which are metabolized by ScrB, and fructose is converted into fructose-6-phosphate by an ATP-dependent ScrK, and the resulting fructose-6-phosphate is metabolized via glycolysis, together with glucose-6-phosphate (J. Biotechnol. (2001) 92:133-158). The sucrose PTS cycle, which functions to convert sucrose into sucrose-6-phosphate and then transports it into the cell, is composed of Enzyme I (EI), histidine protein (HPr), glucose-specific enzyme IIA (EIIAcrr^{Glc}), and sucrose-specific enzyme IIBC (EIIBC^{scr}) (J. Biotechnol, (2001) 92:133-158/J. Biotechnol. (2004) 110:181-199).

Gram-positive microorganisms have a variety of Scr-PTS systems, compared to Gram-negative microorganisms. As the Scr-PTS system of Bacillus subtilis, a sac operon composed of sacA (sucrose-6-phosphate hydrolase), sacP (sucrose-specific EIIBC component), and sacT (transcriptional antiterminator) is well known (J. Bactreriol, (1989) 171: 1519-1523) Corynebacterium glutamicum has an incomplete system, in which it utilizes sucrose via ptsS (sucrose-specific EII component) and scrB (sucrose-6-phosphate hydrolase), but free fructose produced from hydrolysis of sucrose-6-phosphate by ScrB is not utilized within the cell, and transported out of the cell, and thereafter the fructose re-enters the cell via the fructose PTS system (J. Mol. Microbial. Biotechnol. (2007) 12:43-50). Another example of the Scr-PTS system of a Gram-positive microorganism is the scr regulon of Streptococcus mutans. The scr regulon of Streptococcus mutans is composed of 4 genes, scrK (fructokinase), scrA (sucrose-specific EIIABC component), scrB (sucrose-6-phosphate hydrolase) and scrR (LacI-related sucrose-specific repressor), and scrA and scrB are negatively controlled by the ScrR repressor (J. Bacteriol. (2003) 185:5791-5799). The scr regulon of Streptococcus mutans is characterized by possessing a complete Scr-PTS system having even the fructokinase and sucrose transcriptional regulator, unlike Bacillus subtilis or Corynebacterium glutamicum. Therefore, the Scr-PTS system of Streptococcus mutans can be readily introduced into the sucrose non-assimilative strain.

A mechanism of the scr regulon of Streptococcus sp. microorganism is as follows. External sucrose is transported into the cell in the form of sucrose-6-phosphate via ScrA. Sucrose-6-phosphate is then hydrolyzed to glucose-6-phosphate and fructose which are metabolized by ScrB, and fructose is converted into fructose-6-phosphate by an ATP-dependent ScrK, and the resulting fructose-6-phosphate is metabolized via glycolysis, together with glucose-6-phosphate (J. Bacteriol. (1986) 166:426-434, FEMS Microbiol. Lett. (1991) 79:339-346). In particular, most Gram-positive microorganisms including Streptococcus sp. microorganism have different characteristics of ScrA from that of Gram-negative Enterobacteriaceae sp. The ScrA of Enterobacteriaceae sp. retains activities of Enzyme I (EI), histidine protein (HPr), and glucose specific enzyme IIA (EIIAcrr^{Glc}) as a sucrose PTS cycle (J. Biotechnol. (2004) 110:181-199). However, the ScrA of Streptococcus mutans also has a function of enzyme IIA, and thus is not needed to retain the activity of glucose specific enzyme IIA when it is introduced into Escherichia sp. Practically, when the wild-type strains having an inactivated crr gene encoding the glucose specific enzyme IIA are introduced with each of the scr regulons of Streptococcus mutans and Klebsiella pneumoniae and they are spread on a MacConkey agar plate containing 1% sucrose, the strain including the Streptococcus mutans-derived scr regulon shows deep purple colonies, but the strain including the Klebsiella pneumoniae-derived scr regulon does not show deep purple colonies.

The Scr-non PTS system, which requires no PTS for uptake of sucrose into the cell, is exemplified by the well known csc regulon. The csc regulon is mainly derived from a sucrose-assimilative E. coli and exemplified by csc regulon from the wild type E. coli EC3132 (Mol. Gen. Genet. (1992) 235:22-32, U.S. Pat. No. 6,960,455), csc regulon from E. coli KO11 (Biotechnol. Lett. (2004) 26:689-693), csc regulon from the pathogenic E. coli O157:H7 (J. Bacteriol (2002) 184:5307-5316), csc regulon from ATCC13281 (Appl. Microbiol. Biotechnol. (2007) 74:1031-1040) or the like. The csc regulon consists of cscB (proton symport-type sucrose permease), cscK (fructokinase), cscA (sucrose hydrolase), and cscR (LacI-related sucrose-specific repressor), and two operons, cscKB and cscA are negatively controlled by CscR (J. Bacteriol, (2002) 184: 5307-5316).

It was reported that since the Scr-non PTS system is not efficient for uptake of a low level of sucrose, E. coli introduced with the csc regulon has a doubling time of 20 hrs in a medium containing sucrose of 0.2% or less (J. Bacteriol. (2002) 184:5307-5316). Unlike the Scr-non PTS system, the Scr-PTS system allows efficient uptake of even a low level of sucrose into the cell. While the uptake of external sucrose by CscB of the Scr-non PTS system is driven by a hydrogen gradient, the Scr-PTS system requires PEP used as an energy source for the uptake of sucrose into the cell, and thus allows efficient uptake of even a low level of sucrose.

In a specific embodiment of the present invention, the gene encoding a sucrose assimilative microorganism-derived sucrose metabolic enzyme is a gene derived from a microorganism having a sucrose-assimilability, and preferably a gene derived from a microorganism having a PTS-based Scr-PTS system.

In a specific embodiment of the present invention, the gene encoding a sucrose assimilative microorganism-derived sucrose metabolic enzyme may be a gene derived from sucrose assimilative Streptococcus mutans.

In a specific embodiment of the present invention, the gene encoding a sucrose assimilative microorganism-derived sucrose metabolic enzyme may be a gene derived from sucrose assimilative Streptococcus mutans ATCC700610.

In a specific embodiment of the present invention, the gene encoding a sucrose assimilative microorganism-derived sucrose metabolic enzyme may be combinations of the genes encoding fructokinase, sucrose PTS permease, sucrose hydrolase, and sucrose transcriptional regulator, which are derived from Streptococcus mutans.

In a specific embodiment of the present invention, the genes encoding the sucrose assimilative microorganism-derived fructokinase, sucrose PTS permease, sucrose hydrolase, and sucrose transcriptional regulator may be scrK of SEQ ID NO. 4, scrA of SEQ ID NO. 5, scrB of SEQ ID NO. 6, and scrR of SEQ ID NO. 7, respectively.

For the preparation of the microorganism belonging to the genus Escherichia sp. having a sucrose assimilability and an L-amino acid producing ability according to the present invention, introduction of the genes encoding the sucrose assimilative microorganism-derived sucrose PTS permease, sucrose hydrolase, fructokinase, and sucrose transcriptional regulator into the sucrose non-assimilative microorganism belonging to the genus Escherichia sp. may be performed by the method well known in the art.

In a specific embodiment of the present invention, sequences encoding the sucrose PTS permease, the sucrose hydrolase, the fructokinase, and the sucrose transcriptional regulator are introduced into a vector to construct a recombinant vector, and the sucrose non-assimilative Escherichia sp. microorganism having an L-amino acid producing ability is transformed with the constructed recombinant vector so as to prepare an Escherichia sp. microorganism having a sucrose assimilability and an L-amino acid producing ability.

The vector used for the preparation of the Escherichia sp. microorganism of the present invention is not particularly limited, and any known expression vectors may be used. Preferably, pACYC177, pACYC184, pCL, pECCG117, pUC19, pBR322, or pMW118 may be used.

As used herein, the term “transformation” means method in which a gene is introduced into a host cell to be expressed in the host cell. The transformed gene, if it can be expressed in the host cell, may be inserted in the chromosome of the host cell or may exist independent of the chromosome. In addition, the transformed gene is defined as a polynucleotide capable of encoding a polypeptide, and includes DNA and RNA. The transformed gene may be in a suitable form that can be introduced into the host cell and expressed therein. For example, the transformed gene may be introduced into the host cell in the type of expression cassette which is a polynucleotide expressome including whole elements for expressing the gene by itself. Typically, the expression cassette includes a promoter, a transcription termination signal, a ribosome binding site and a translation termination signal, which are operably linked to the transformed gene. The expression cassette may be in the type of the expression vector capable of self-replication. The transformed gene may also be introduced into the host cell by itself or in the type of polynucleotide expressome so as to be operably linked to the sequence required for expression in the host cell.

In a specific embodiment of the present invention, the sucrose non-assimilative Escherichia sp. microorganism having an L-amino acid producing ability may be transformed with a recombinant vector harboring a gene encoding a Streptococcus mutans-derived Scr-PTS enzyme in order to acquire a sucrose assimilability.

In a specific embodiment of the present invention, the sucrose non-assimilative Escherichia sp. microorganism having an L-amino acid producing ability may be transformed with a recombinant plasmid including a sequence of SEQ ID NO. 8 in order to acquire a sucrose assimilability. Specifically, the recombinant plasmid of SEQ ID NO. 8 includes Streptococcus mutans (ATCC700610)-derived scrKABR, namely, the fructokinase-encoding scrK of SEQ ID NO. 4, the sucrose PTS permease-encoding scrA of SEQ ID NO. 5, the sucrose hydrolase-encoding scrB of SEQ ID NO. 6, and the sucrose transcriptional regulator-encoding scrR of SEQ ID NO. 7.

The microorganism belonging to the genus Escherichia sp. having a sucrose assimilability and an L-amino acid producing ability according to the present invention is a microorganism belonging to the genus Escherichia sp. that is able to produce L-amino acid and retains the activities of sucrose PTS permease, sucrose hydrolase, fructokinase, and sucrose transcriptional regulator at the same time, so as to utilize sucrose as a carbon source, and preferably Escherichia coli.

In a specific embodiment of the present invention, the L-amino acid may be L-threonine, O-succinyl-homoserine, O-acetyl-homoserine, L-methionine, L-lysine, L-homoserine, L-isoleucine, L-valine, or L-tryptophan.

In a specific embodiment of the present invention, the L-amino acid may be L-threonine.

In a specific embodiment of the present invention, the microorganism belonging to the genus Escherichia sp. having a sucrose assimilability and an L-amino acid producing ability may be Escherichia coli CA03-0208 (KCCM 10994) that is obtained by transforming Escherichia coli ABA5G having an L-threonine-producing ability with a vector having the sequence of SEQ ID NO. 8 including the Streptococcus mutans-derived scrKABR gene cluster.

Further, the present invention provides a method for producing an L-amino acid, comprising the steps of culturing the microorganism belonging to the genus Escherichia sp. having a sucrose assimilability and an L-amino acid producing ability in a medium containing sucrose as a carbon source; and recovering an L-amino acid from the culture medium.

In a specific embodiment of the present invention, the L-amino acid may be L-threonine, O-succinyl-homoserine, O-acetyl-homoserine, L-methionine, L-lysine, L-homoserine, L-isoleucine, L-valine, or L-tryptophan.

In a specific embodiment of the present invention, the L-amino acid may be L-threonine.

The method for producing an L-amino acid according to the present invention includes the step of culturing the microorganism belonging to the genus Escherichia sp. Having a sucrose assimilability and an L-amino acid producing ability.

In a specific embodiment of the present invention, the step of culturing the Escherichia sp. microorganism may be conducted in a medium and under culture conditions that are suitable for the corresponding microorganism. The medium and culture conditions suitable for the corresponding microorganism can be readily selected and adjusted by any person skilled in the art to which it pertains. Examples of the culturing method include batch type, continuous type and fed-batch type manners, but are not limited thereto.

In a specific embodiment of the present invention, the step of culturing the microorganism belonging to the genus Escherichia sp. may be conducted by culturing the strain in a typical medium that is supplemented with appropriate carbon sources including sucrose, nitrogen sources, amino acids, and vitamins under aerobic conditions and temperature or pH control.

The medium used in the present invention includes sucrose or molasses containing a high concentration of sucrose as a main carbon source, and may include various carbon sources in addition to the main carbon source. The nitrogen source included in the medium may be used either singly or in combinations of organic nitrogen sources such as peptone, yeast extract, broth, malt extract, corn steep liquor, and soy bean, and inorganic nitrogen sources such as urea, ammonium sulfate, ammonium chloride, ammonium phosphate, ammonium carbonate and ammonium nitrate. In the medium, phosphorus sources such as potassium dihydrogen phosphate, dipotassium hydrogen phosphate or corresponding sodium-containing salts may be included. In addition, the medium may be supplemented with amino acids, vitamins, and appropriate precursors. These main components may be added to the media in a batch type or a continuous type.

During cultivation, compounds such as ammonium hydroxide, potassium hydroxide, ammonia, phosphoric acid, and sulfuric acid may be properly added so as to adjust the pH of the cultures. During cultivation, defoaming agents such as fatty acid polyglycol ester may be used so as to prevent the formation of foams. Generally, the cultivation temperature may be maintained at 27° C. to 37° C., and preferably at 30° C. to 35° C. The cultivation may be continued as long as a production amount of the desired material, L-amino acid is increased under the given conditions, and for example, for 10 to 100 hrs.

The method for producing an L-amino acid according to the present invention includes the step of recovering the L-amino acid from the culture of the microorganism. The method of recovering the L-amino acid from the culture may be performed by a proper method known in the art, depending on the culturing procedures, for example, batch type, continuous type or fed-batch type.

Hereinafter, the present invention will be described in more detail with reference to Examples. However, these Examples are for illustrative purposes only, and the invention is not intended to be limited by these Examples.

### Example 1

### Cloning and Identification of Sequence of scr Regulon which is Involved in Sucrose Assimilation

(1) Cloning of Sucrose Assimilative Microorganism-Derived scr Regulon

To impart a sucrose assimilability to a sucrose non-assimilative Escherichia sp. microorganism, the gene cluster involved in sucrose assimilation, scr regulon was obtained from a sucrose assimilative microorganism, Streptococcus mutans. The scr regulon of Gram-positive Streptococcus mutans is composed of four genes, scrK (fructokinase), scrA (sucrose-specific EIIBC component), scrB (sucrose-6-phosphate hydrolase), and scrR (LacI-related sucrose-specific repressor), and two operons, scrA and scrB are negatively controlled by the ScrR repressor (J. Bacteriol. (2003) 185:5791-5799). In particular, the scr regulon of Streptococcus mutans is characterized by possessing a complete Scr-PTS system having the fructokinase and sucrose transcriptional regulator, unlike Bacillus subtilis or Corynebacterium glutamicum.

The Scr-PTS system, scrKABR genes were obtained by PCR (Polymerase Chain Reaction) using the chromosome of Streptococcus mutans (ATCC700610D-5) purchased from American Type Culture Collection as a template.

The scrKABR gene that is the scr regulon Streptococcus mutans was amplified by PCR using a pair of primers of SEQ ID NO. 1 and SEQ ID NO. 2, so as to obtain four types of genes, which are consecutively present on the genome, as a single polynucleotide. The primer of SEQ ID NO. 1 has the ApaLI restriction site, and the primer of SEQ ID NO. 2 has the StuI restriction site. The primers that were used were prepared based on information on scrKABR and its surrounding sequence of Streptococcus mutans (KEGG organism, smu) available in the KEGG (Kyoto Encyclopedia of Genes and Genomes).

PCR was performed under the conditions including denaturation at 94° C. for 3 min, 25 cycles of denaturation at 94° C. for 30 sec, annealing at 56° C. for 30 sec, and polymerization of at 72° C. for 5 min, and then polymerization for at 72° C. for 7 min. As a result, a polynucleotide of 6480 bp was obtained from Streptococcus mutans. The polynucleotide obtained by PCR was treated with ApaLI and FspI, and then cloned into the ApaLI and FspI sites of a pACYC177 vector. Thereafter, E. coli DH5α was transformed with the vector, and spread on a MacConkey agar plate containing 1% sucrose. Among the colonies, deep purple colonies were selected, and then a plasmid derived from Streptococcus mutans was obtained using a typical plasmid miniprep.

(2) Identification of Sequence Determination of scrKABR Gene

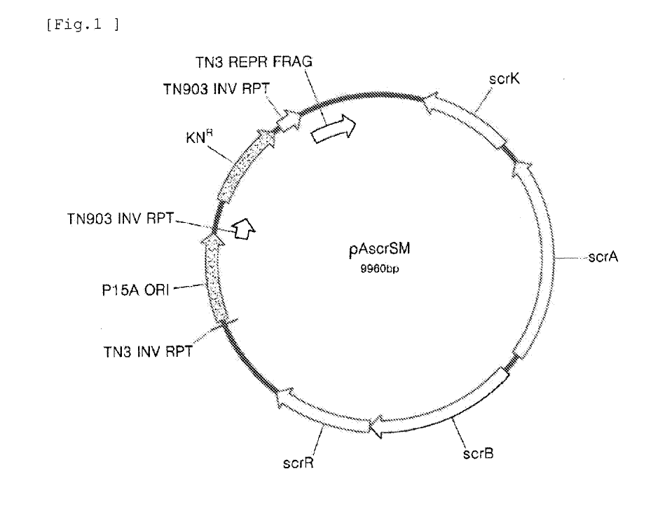

The plasmid containing the scr regulon of Streptococcus mutans obtained in (1) was designated as pAscrSM, and the sequence (SEQ ID NO. 3) of scrKABR cloned into the ApaLI and FspI sites was determined by a sequence determination method typically used in the art. FIG. 1 shows the construction of a recombinant plasmid pAscrSM containing Streptococcus mutans-derived scrKABR. In the scrKABR sequence of SEQ ID NO. 3, the position from 392 to 1273 was identified as the scrK gene (SEQ ID NO. 4), the position from 1473 to 3467 was identified as the scrA gene (SEQ ID NO. 5), the position from 3663 to 5102 was identified as the scrB gene (SEQ ID NO. 6), and the position from 5105 to 6067 was identified as the scrR gene (SEQ ID NO. 7).

### Example 2

### Construction of Sucrose Assimilative, L-Amino Acid Producing Microorganism

(1) Transformation with Recombinant Plasmid

In order to examine whether a threonine-producing E. coli grows using sucrose and produces threonine efficiently, when the E. coli is transformed with the gene cluster involved in sucrose assimilation, scr regulon-containing pAscrSM (SEQ ID NO. 8) obtained in Example 1, the plasmid was introduced into E. coli ABA5G by a typical transformation method. The E. coli ABA5G transformed with pAscrSM was spread on a MacConkey agar plate containing 1% sucrose. Among the colonies, deep purple colonies were selected. PCR was performed to confirm that the selected colonies had the sucrose assimilation related gene-containing plasmid.

(2) Production of Threonine by Microorganism Transformed with pAscrSM

The colony obtained in (1) was cultured on a LB solid medium (1 g of tryptone, 1 g of NaCl, 0.5 g/100 ml of yeast extract, 1.5% agar) in a 33° C. incubator overnight. One loop of the cultured strain was inoculated in 25 mL of a titration medium having the composition of the following Table 1 and sucrose as a main carbon source, and then cultured in a 33° C. incubator at 200 rpm for 90 hrs,

TABLE 1

| | Concentration | |
|---|---|---|
| Composition | (per liter) | |
| Sucrose | 70 | g |
| KH₂PO₄ | 2 | g |
| (NH₄)₂SO₄ | 25 | g |
| MgSO₄•7H₂O | 1 | g |
| FeSO₄•7H₂O | 5 | mg |
| MnSO₄•4H₂O | 5 | mg |
| Yeast extract | 2 | g |
| Calcium carbonate | 30 | g |

| | |
|---|---|
| pH | 6.8 |

As a control group, the parental strain E. coli ABA5G transformed with no plasmid was used, and cultured in the medium having the composition of Table 1 using glucose instead of sucrose, in order to compare the sucrose utilization rate to the glucose utilization rate and threonine productivity. The results are summarized in the following Table 2,

TABLE 2

| | Glucose (70 g/L) | | Sucrose (70 g/L) | |
|---|---|---|---|---|
| | OD | L-threonine (g/L) | OD | L-threonine (g/L) |
| ABA5G | 14.2 | 21.2 | — | — |
| ABA5G/pAscrSM | 12.1 | 17.4 | 13.2 | 21.7 |

As shown in Table 2, the pAscrSM-harboring E. coli ABA5G/pAscrSM produced 17.4 g/L of L-threonine in the titration medium containing glucose, and produced 21.7 g/L of L-threonine in the titration medium containing sucrose during 90 hr cultivation. On the contrary, the parental strain E. coli ABA5G transformed with no plasmid produced 21.2 g/L of L-threonine by utilizing glucose during 90 hr cultivation, but did not grow in the medium containing sucrose as a sole carbon source. This result indicates that the sucrose non-assimilative E. coli ABA5G having an L-threonine producing ability is introduced with pAscrSM so as to have sucrose assimilability, and therefore it utilizes sucrose to have L-threonine productivity equivalent to or better than that of the parental strain E. coli ABA5G utilizing the glucose titration medium.

Therefore, the pAscrSM-harboring recombinant strain showed excellent sucrose utilization and L-threonine productivity, and thus the transformed microorganism was designated as CA03-0208, deposited in the international depository authority, Korean Culture Center of Microorganisms, which is the Subsidiary Culture Collection of the Korean Federation of Culture Collections, (located at 361-221, Hongje-1-dong, Seodaemon-gu, Seoul, Korea) on Feb. 23, 2009, and assigned accession number KCCM 10994.

It will be apparent to those skilled in the art that various modifications and changes may be made thereto without departing from the scope and spirit of the invention. Therefore, it should be understood that the above embodiments are not limitative, but illustrative in all aspects.

The sequences of SEQ ID NOs. 1 to 8 described herein are listed in the accompanying sequence listing.

### EFFECT OF THE INVENTION

The microorganism having a sucrose assimilability and an L-amino acid producing ability according to the present invention is used to economically produce an L-amino acid using inexpensive sucrose as a carbon source.

What is claimed is:

1. A microorganism belonging to the genus Escherichia sp. having a sucrose assimilability and an L-amino acid producing ability, which is obtained by introducing Streptococcus mutans-derived genes encoding fructokinase, sucrose PTS permease, sucrose hydrolase, and sucrose transcriptional regulator into a sucrose non-assimilative microorganism belonging to the genus Escherichia sp. having an L-amino acid producing ability.

2. The microorganism belonging to the genus Escherichia sp. according to claim 1, wherein the genes encoding fructokinase, sucrose PTS permease, sucrose hydrolase, and sucrose transcriptional regulator are scrK of SEQ ID NO. 4, scrA of SEQ ID NO. 5, scrB of SEQ ID NO. 6, and scrR of SEQ ID NO. 7, respectively.

3. The microorganism belonging to the genus Escherichia sp. according claim 2, wherein the microorganism belonging to the genus Escherichia sp. is obtained by transforming the sucrose non-assimilative microorganism belonging to the genus Escherichia sp. with a recombinant vector of SEQ ID NO. 8.

4. The microorganism belonging to the genus Escherichia sp. according to claim 1, wherein the microorganism belonging to the genus Escherichia sp., is E. coli.

5. The microorganism belonging to the genus Escherichia sp. according to claim 4, wherein the E. coli is E. coli CA03-0208 (KCCM 10994).

6. The microorganism belonging to the genus Escherichia sp. according to claim 1, wherein the L-amino acid is L-threonine.

7. A method for producing an L-amino acid, comprising the steps of culturing the microorganism belonging to the genus Escherichia sp. as in one of claims 1-6 in a medium containing sucrose as a carbon source; and recovering an L-amino acid from the culture medium.

8. The method according to claim 7, wherein the L-amino acid is L-threonine.
